(19) United States
(12) Patent Application Publication
(10) Pub. No.: US 2025/0110128 A1
(43) Pub. Date: Apr. 3, 2025

(54) METHOD FOR DETECTING BIOLOGICAL OBJECTS BY MEANS OF SURFACE PLASMON RESONANCE IMAGING

(71) Applicants: ARYBALLE, GRENOBLE (FR); COMMISSARIAT À L'ENERGIE ATOMIQUE ET AUX ENERGIES ALTERNATIVES, PARIS (FR); CENTRE NATIONAL DE LA RECHERCHE SCIENTIFIQUE, PARIS 16 (FR); UNIVERSITE GRENOBLE ALPES, Saint Martin d'Heres (FR)

(72) Inventors: Cristina-Cassiana ANDREI, GRENOBLE (FR); Cyril HERRIER, GRENOBLE (FR); Sami SLIMANI, GRENOBLE (FR); Yanxia HOU-BROUTIN, GRENOBLE (FR); Christophe MOREAU, GRENOBLE CEDEX 9, (FR); Thierry LIVACHE, GRENOBLE (FR)

(21) Appl. No.: 18/727,911

(22) PCT Filed: Jan. 11, 2023
(86) PCT No.: PCT/EP2023/050532

§ 371 Date: Jul. 10, 2024

(30) Foreign Application Priority Data
Jan. 14, 2022 (FR) FR2200324

(51) Int. Cl. G01N 33/569; G01N 21/552

(52) U.S. Cl. CPC G01N 33/56983; G01N 21/553; G01N 33/56916; G01N 2333/165; G01N 2469/10

(57) ABSTRACT

A method for detecting biological objects by means of an SPR imaging detection system comprising an optical measurement device configured to generate plasmon resonance on a functionalized surface when said surface is exposed to a gas; the method comprising: an assimilation step by exposing the functionalized surface to a sample of interest formed of an aqueous carrier liquid containing the biological objects; a step of removing the liquid in contact with the functionalized surface and exposing the surface to a gas not containing the biological objects, said objects remaining bound to the ligands of the sensitive site of the functionalized surface; a step of acquiring an image of the sensitive site; a step of detecting the biological objects from the acquired image.

### TECHNICAL FIELD

The subject of the invention is detecting biological objects using a surface plasmon resonance imaging detection system. Biological objects can be small, for example of the order of a hundred nanometers to a few micrometers, particularly with regard to the spatial resolution of the detection system.

### PRIOR ART

The ability to detect biological objects, especially small ones, such as microorganisms like viruses or bacteria, is an increasingly important issue, particularly in the healthcare sector, as well as in the agri-food and environmental sectors. Detecting such biological objects can be carried out using a Surface Plasmon Resonance Imaging (SPRi) detection system, which has the advantage of being a label-free detection technique in which the biological objects are not pre-labeled with a developer.

Surface plasmon resonance occurs when a light signal illuminates a metal-dielectric interface under certain conditions of wavelength, polarization and angle of incidence. This interface can be formed by a thin metal layer on the surface of a prism, and by the fluid containing the analytes. It can include ligands adapted to bind specifically to analytes, thus forming a functionalized surface. When these conditions are met, free electrons on the surface of the metal layer absorb incident photons and convert them into surface plasmon waves. These plasmonic resonance conditions depend in particular on the refractive index at the surface of the metal layer. Thus, when an adsorption/desorption interaction occurs between a ligand and an analyte, the refractive index changes and plasmon resonance conditions are modified. This makes it possible to monitor adsorption/desorption interactions in real time, without the need for labeling.

FIG. 1A is a schematic and partial view of an SPR imaging detection system 1 according to a prior art example described in particular in document WO2018/158458A1. It comprises a functionalized surface 5, located on one face of a prism 4, comprising a plurality of sensitive sites adapted to capture biological objects present in a liquid sample by adsorption, and an optical measuring device 10 adapted to acquire an image of the sensitive sites. It also includes a processing unit 6 for detecting the presence of biological objects, for example, on the basis of images supplied by the optical measuring device 10. A fluid management device (not shown) can be provided to bring the liquid sample into contact with the functionalized surface 5. The optical measuring device 10 comprises an optical source 11, an optical shaping device formed here by a collimating lens 12 and a polarizer 13, an optical imaging device 14 and a matrix photodetector 15 (image sensor).

FIGS. 1B and 1C are schematic and partial views of an example of the functionalized surface 5, in perspective (FIG. 1B) and cross-section (FIG. 1C). The functionalized surface 5 here is a surface of a metal layer 3 that coats one face of the prism 4. Ligands adapted to adsorb biological objects are arranged in several distinct zones, which then form the sensitive sites (or probes) of the functionalized surface 5. The ligands may be identical or different from one sensitive site to another.

FIG. 1D shows an example of an SPR curve, i.e. an evolution of the reflectivity R as a function of an angle of incidence θ of the excitation signal on the functionalized surface, here in the context of a so-called reflectivity interrogation. The optical source emits an excitation signal illuminating the sensitive site at an angle of incidence known as the working angle θ_{R}, allowing surface plasmons to be generated, thus optimizing the sensitivity of the detection system. The reflectivity R is determined, i.e. the ratio of the intensity of the measurement signal received to the intensity of the excitation signal emitted. The value of reflectivity R depends locally on the optical index of the functionalized surface, which in turn depends on the surface plasmons generated and the amount of adsorbed material, which varies over time as a result of adsorption/desorption interactions with the ligands.

FIG. 1E shows an example of the temporal evolution of reflectivity R (also called sensorgram). The optical measuring device is pre-configured to generate a surface plasmon resonance at the functionalized surface when said surface is exposed to a liquid (so-called liquid phase configuration, or ‘liquid mode’). The working angle θ_{R} is defined in an angular range where the sensitivity of the detection system is optimal. In a first step, a reference liquid is injected, for example a buffer solution not containing the biological objects. Reflectivity R then has an initial value Rᵢ. In a second step, a liquid sample formed from the buffer solution now containing the biological objects is injected. The latter then bind to the ligands by adsorption, causing a variation in the optical index at the surface of the sensitive site, resulting in an increase in reflectivity to a stationary value R_{f}. Adsorbed biological objects can then be characterized by the value of the variation in induced reflectivity ΔR.

However, it appears that the detection of biological objects is particularly difficult, and requires the use of a high-performance detection system, particularly in terms of sensitivity and spatial resolution. In fact, biological objects may have a refractive index close to that of the buffer solution containing them, which means that the detection system must be highly sensitive. In addition, they may be small, for example in the order of a micron, making it difficult to detect individual biological objects adsorbed on the functionalized surface, and requiring the detection system to have high spatial resolution.

The paper by Laplatine et al. entitled Spatial resolution in prism-based surface plasmon resonance microscopy, Opt. Express 22 (19) 22771-22785 (2014) indicates that spatial resolution depends on the propagation length of plasmonic waves, but also on optical aberrations linked to the transmission of optical signals in the prism. It proposes a detection system optimized for spatial resolution by the use of an optimized prism, an imaging system including a magnification lens, and image plane reconstruction by scanning and image processing. The spatial resolution obtained is then equal to 1.7 μm and 2.8 μm along axes respectively perpendicular and parallel to the direction of plasmonic wave propagation, over a wide field of observation.

Furthermore, the article by Boulade et al. entitled Early detection of bacteria using SPR imaging and event counting: experiments with Listeria monocytogenes and Listeria innocua, RSC Adv., 2019, 9, 15554 describes the use of an SPR detection system with optimized spatial resolution of the order of 6 μm along an axis parallel to the direction of plasmonic wave propagation. Bacteria detection is carried out, allowing for each adsorbed bacterium to be identified, with a size of the order of magnitude of the spatial resolution.

As a result, detecting small biological objects by SPR imaging is problematic due to the spatial resolution of the detection system. There is therefore a need to be able to detect each and every biological object adsorbed on sensitive sites, in particular to count these biological adsorption events, without having to resort to a detection system made complex to optimize spatial resolution while maintaining a wide field of observation (1 to 100 mm² for example). There is also a need to be able to detect biological objects of small size in terms of spatial resolution.

### DISCLOSURE OF THE INVENTION

The aim of the invention is to at least partially overcome the drawbacks of the prior art, and more particularly to provide a method for detecting biological objects using a conventional SPR imaging detection system in the sense that it is not necessary to resort to a complex detection system to optimize spatial resolution. The detection method can also be used to identify biological objects of small size in terms of spatial resolution.

To this end, the object of the invention is a detection method, by means of a surface plasmon resonance imaging detection system comprising: a functionalized surface having at least one sensitive site formed of ligands adapted to bind to biological objects; and an optical measuring device configured to generate surface plasmon resonance at the functionalized surface when said surface is exposed to a gas, and adapted to acquire an image of the sensitive site. The method comprises:
an assimilation step, involving exposure of the functionalized surface to a sample of interest formed of an aqueous carrier liquid containing the biological objects, the biological objects then binding to the ligands;
a step for acquiring an image of the sensitive site by the optical measuring device;
a step of detecting biological objects from the acquired image.

According to the invention, the method comprises a step, carried out between the assimilation step and the acquisition step, of removing the liquid in contact with the functionalized surface, and exposing the functionalized surface to a gas not containing the biological objects, the biological objects remaining bound to the ligands, the acquisition step then being carried out while the biological objects are bound to the ligands and the gas is in contact with the functionalized surface.

Some preferred but non-limiting aspects of this detection method are as follows.

Biological objects can be smaller, preferably up to 200 times smaller, than a predefined spatial resolution of the detection system.

Biological objects can be between 50 nm and 50 μm in size.

The detection system can have a spatial resolution of at least 5 μm.

The detection method can include a step for rinsing the functionalized surface with at least one liquid, carried out between the assimilation step and the removal step.

During the fluid injection step (assimilation step), the liquid carrying the sample of interest may be in a continuous or dispersed phase.

During the removal step, the gas may have a relative humidity of at least 50%.

Biological objects can be selected from viral particles, bacteriophages, bacteria and their spores, archaea, microscopic fungi and their spores, unicellular protozoa, blood or non-circulating cells, circulating vesicles, exosomes, pollens, biological objects comprising a synthetic particle to which at least one ligand or biological protein is attached.

### BRIEF DESCRIPTION OF THE DRAWINGS

Other aspects, purposes, advantages and features of the invention will become clearer from the following detailed description of preferred embodiments thereof, given by way of non-limiting example, and made with reference to the appended drawings, in which:

FIG. 1A, already described, is a partial schematic cross-sectional view of an SPR imaging detection system according to an example of the prior art;

FIGS. 1B and 1C, already described, are schematic and partial views, in perspective and cross-section, of the functionalized surface of the detection system of FIG. 1A;

FIG. 1D, already described, shows an example of SPR curves, i.e. changes in reflectivity R as a function of the angle of incidence θ of the excitation signal;

FIG. 1E, already described, illustrates an example of sensorgram, i.e. a temporal evolution of reflectivity R, during a preliminary configuration in ‘liquid mode’ of the optical measuring device of the detection system, and during an assimilation step of a biological object detection method;

FIG. 2 is a partial schematic view of a detection system used in a detection method according to one embodiment;

FIGS. 3A and 3B each illustrate a functionalized surface and an SPR curve (in this case, the evolution of reflectivity R as a function of the local refractive index n), during a prior configuration of the optical measuring device in ‘liquid mode’ (FIG. 3A), and during an assimilation step (FIG. 3B) of a detection method in which it is necessary to use a detection system optimized for spatial resolution;

FIG. 4A to 4D each illustrate a functionalized surface and an SPR curve (here, the evolution of reflectivity R as a function of local refractive index n), associated with a detection system with standard performance in terms of spatial resolution and here configured in ‘gas mode’ (FIG. 4A), and for different steps of a detection method according to a first embodiment, namely: an assimilation step (FIG. 4B), a rinsing step (FIG. 4C), and a step of removal and acquisition of a detection image (FIG. 4D);

FIG. 5A to 5D each illustrate a functionalized surface and an SPR curve (here, the evolution of reflectivity R as a function of local refractive index n), associated with a detection system with standard performance in terms of spatial resolution and here configured in ‘gas mode’ (FIG. 5A), and for various steps of a detection method according to a second embodiment, namely: an assimilation step (FIG. 5B), a rinsing step (FIG. 5C), and a step of removal and acquisition of a detection image (FIG. 5D);

FIG. 6A to 6C illustrate images acquired during different steps of the detection method according to the first embodiment, namely: before the assimilation step (FIG. 6A), during the rinsing step following the assimilation step (FIG. 6B), and during the removal and detection image acquisition step (FIG. 6C), where the biological objects are SARS-COV-2 viral particles;

FIG. 7A illustrates a detection image acquired as part of the detection method according to the first embodiment, where the biological objects are SARS-COV-2 virus particles; and FIG. 7B to 7D illustrate sensitive sites whose ligands are of different types;

FIG. 8A to 8C each illustrate a sensitive site of a detection image acquired as part of the detection method according to the first embodiment, for different types of biological objects, namely modified polystyrene beads (FIG. 8A), SARS-COV-2 virus particles (FIG. 8B), and bacteria (FIG. 8C).

### DETAILED DESCRIPTION OF EMBODIMENTS

In the figures and in the rest of the description, the same references represent identical or similar elements. In addition, the various elements are not shown to scale, for the sake of clarity. Furthermore, the various embodiments and variants are not mutually exclusive and can be combined with one another. Unless otherwise indicated, the terms “substantially”, “approximately”, “of the order of” mean to within 10%, and preferably to within 5%. Furthermore, the words “between . . . and . . . ” and equivalents mean that bounds are included, unless otherwise stated.

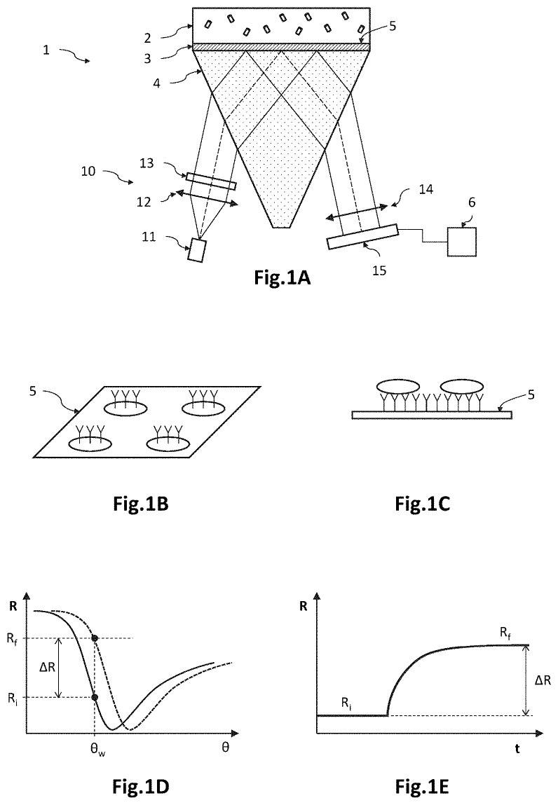

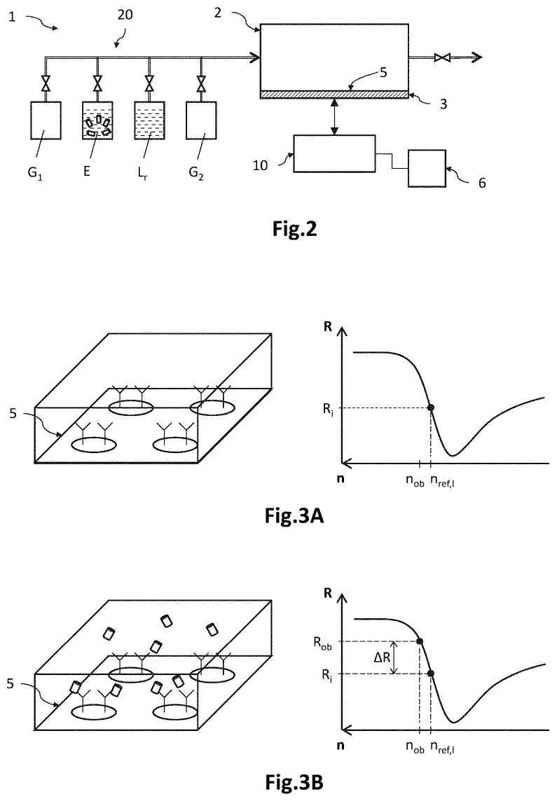

FIG. 2, in connection with FIGS. 1A and 1B already briefly described, illustrates a detection system 1 used as part of a detection method according to one embodiment. The detection system 1 therefore comprises a functionalized surface 5 located here in a measuring chamber 2, an optical measuring device 10, possibly a processing unit 6, and possibly a fluidic management device 20.

As will be explained in greater detail below, the detection method described in the invention allows the use of a detection system 1 with standard performance, particularly in terms of spatial resolution, and enables the detection of each of the biological objects adsorbed on the functionalized surface 5, even though they may be smaller in size than the spatial resolution. The adsorbed biological objects can then be counted, and possibly characterized in terms of their affinity for the various ligands present.

Spatial resolution is defined as the minimum distance between two points on the functionalized surface that can be distinguished by the detection system 1. In particular, it is limited by the propagation length Lx of the plasmonic wave and by any optical aberrations associated with the transmission of optical signals through the prism of the optical measuring device 10. Standard detection systems generally have a spatial resolution of around 5 to 10 μm. These SPR imaging detection systems can be optimized for spatial resolution, as described in the article by Laplatine et al. 2014 mentioned above and thus offer a spatial resolution of the order of a few microns. The detection method implemented according to the invention allows the use of standard detection systems, even though the aim is to detect biological objects whose size may be much smaller than the spatial resolution.

The biological objects to be detected here are natural biological objects such as viral particles such as SARS-COV-2, complete or incomplete, bacteriophages, bacteria, bacterial spores, archaea, microscopic fungi (yeasts and molds) and their spores, single-cell protozoa, blood or non-circulating cells, circulating vesicles, exosomes, pollens, synthetic biological objects such as nano or microparticles decorated with ligands or biological proteins (e.g. biotinylated particles coated with antibodies). They may be prokaryotic or eukaryotic organisms, single or multi-cellular, of plant, animal or human origin. The microorganism may be alive, i.e. capable of multiplying.

Biological objects can have a size of the order of a few tens of nanometers to a few tens of microns, for example between around 50 nm and around 50 μm, and for example between 100 nm and 10 μm. The size of a biological object is defined as its maximum dimension, for example its diameter when it has a circular shape, or its large dimension when it has an elongated shape. In the case of viruses, size can be in the order of a hundred nanometers, and in the case of bacteria, in the order of a micrometer.

The size of the biological objects may be smaller than, approximately equal to or even greater than the spatial resolution of the detection system 1. Preferably, the biological objects have a size smaller than the spatial resolution, preferably at most 200 times smaller or at most 100 times smaller, or even at most 50 or 10 times smaller. By way of example, in the case of a spatial resolution equal to around 10 μm (along the axis of propagation of the plasmonic wave), the size of the biological objects can be between around 50 nm and 10 μm, preferably between 100 nm, 200 nm, 500 nm, 1 μm . . . and 10 μm. As detailed below, despite the fact that the size of the biological objects may be smaller than the spatial resolution, the detection method according to the invention allows the use of a detection system which is standard in terms of spatial resolution, i.e. which does not necessarily have an optimized spatial resolution.

Biological objects are contained in the aqueous carrier liquid of a sample of interest, and not in a gaseous sample without a carrier liquid. The carrier liquid can in particular be a buffer solution. More broadly, the sample of interest may be a biological sample (from living or previously living organisms), a food sample (from food), a water sample (waste water, fresh water, etc.), or culture fluids from viruses or microorganisms, among others. The liquid carrying the sample of interest may be in a continuous phase, so that the sample of interest is a liquid sample, or it may be in a dispersed phase as droplets containing the biological objects located in a liquid dispersing phase (the sample is then said to be a liquid) or a gas phase (the sample is then said to be a mist or aerosol).

The detection system 1 thus comprises a functionalized surface 5 of a metal layer 3 (homogeneous or not), formed of at least one sensitive site (probe), and here of a plurality of sensitive sites distinct from one another. Here, the functionalized surface 5 is located on an upper face of a prism 3. The sensitive sites contain ligands capable of interacting with the biological objects to be detected. The sensitive sites may be identical or different from one another, in terms of the affinity of the ligands with the biological objects to be detected. The functionalized surface 5 can be passivated by methods known to those skilled in the art, in particular using PEG (polyethylene glycol) or proteins such as bovine serum albumin. Here, the functionalized surface 5 is located in a measuring chamber 2, but alternatively it may not be located in a fluidic chamber, and thus be exposed to a surrounding gas.

Ligands are adapted to specifically capture (bind) the biological objects to be detected. See WO2012073202A1 for examples of ligands that can be used to bind to biological objects. These may include natural receptors for biological objects, proteins, bacteriophages or whole viruses that may be inactivated, immunoglobins such as antibodies and their fragments, synthetic compounds, and more. They can also be DARpins of different sizes and composed of natural or unnatural amino acids, with or without the addition of biological molecules, such as small molecules, peptides, proteins, polysaccharides, lipids, chemical groups or particles. They can also be receptors modified by chimeric approaches, such as fusion to the Fc domain of immunoglobulins, by mutagenesis, by insertion of non-natural amino acids, or by addition of chemical groups or particles. They can also be synthetic peptides fixing the biological object, including natural or non-natural amino acids, with or without the addition of chemical groups or particles. It may also be a substrate cleavable by the microorganism or by another physicochemical stimulus, containing or not containing additional domains, molecules or particles, including peptide sequences, lipids, polysaccharide chains or peptidoglycans. It can also be living or fixed cells.

Furthermore, the ligands have a sufficiently strong affinity for the biological objects to ensure that the latter remain bound to the ligands during a step of removing the liquid present in contact with the functionalized surface, possibly preceded by a step of rinsing said surface. It should be remembered that the interaction between an analyte A (here a biological object) and a ligand L is a reversible phenomenon that can be described by the Langmuir model, and is characterized by an association constant kₐ associated with the association of analyte A with ligand L to form a compound LA (ligand-analyte), and by a dissociation constant kd associated with the dissociation of compound LA. This relationship is expressed as follows:

$$A + L {{⟶}^{⟵}}_{{k}_{d}}^{{k}_{a}} AL \qquad [ Math 1 ]$$

In the context of the invention, the affinity between the biological objects and the ligands is sufficiently strong for the biological objects to remain bound to the ligands during the rinsing and removal steps of the liquid present on the functionalized surface 5. In other words, the ratio kₐ/k_{d} is greater than unity, and preferably very large. Furthermore, a biological object can be bound to several ligands at the same sensitive site, increasing its adhesion to the functionalized surface 5.

Some sensitive sites may contain receptors of a similar nature to the ligands but with no affinity to the biological objects, thus serving as negative controls. In other words, these non-specific receptors on biological objects can be used to determine measurement noise, or even probe drift, and thus contribute to validating the measurement signals received.

The optical measuring device 10 is adapted to illuminate the functionalized surface 5 with an excitation signal so as to generate surface plasmons, and to receive a measurement signal reflected by the functionalized surface 5 and acquire an image thereof and more precisely of the sensitive sites. Image acquisition can be performed in real time, at a high acquisition frequency, or can be performed when the association regime of biological objects with ligands is deemed to have reached a steady state.

Thus, the optical measuring device 10 comprises an optical source 11 adapted to transmit the excitation optical signal in the direction of the sensitive sites, with a predefined wavelength, polarization and angle of incidence, and thus to generate surface plasmons at the functionalized surface 5. The optical source 11 may comprise a light-emitting diode, preferably monochromatic, or a laser diode. It also includes optical elements for shaping the optical excitation signal, such as one or more collimating lenses 12, and a polarizer 13.

The optical measuring device 10 may also include optical elements (optical imaging device 14) for conjugating the functionalized surface to the receiving face of a matrix photodetector, thus enabling the functionalized surface 5 to be imaged onto the receiving plane of the matrix photodetector 15.

The optical measuring device 10 includes an image sensor 15, i.e. a matrix photodetector for acquiring the image of the functionalized surface 5, and more precisely that of the sensitive sites. In this way, the light beams of the measurement signal coming from the sensitive sites are detected together and in real time, in the form of an image acquired by the same image sensor 15. Image sensor 15 can be a CCD or CMOS sensor. It comprises a matrix of pixels whose spatial resolution is such that several pixels acquire the measurement signal from the same sensitive site. By way of example, a sensitive site dimension of around 300 μm can be covered by around 150 pixels.

The detection system 1 can include a processing unit 6, connected to the optical measurement device 10, for optionally processing the images acquired by the image sensor 15 to facilitate detection of the biological objects, for example by filtering to improve the sharpness of the acquired images, or even for averaging several elementary images to form a final image from which the biological objects are detected, as described in particular in application WO2020/141281A1. The processing unit 6 can also be connected to the fluid management device, to implement at least some of the steps in the detection method.

The detection method according to the invention comprises successive steps of exposing the functionalized surface 5 to different fluids. Exposure means that the fluid comes into contact with the functionalized surface 5. These exposure steps can be carried out in a controlled manner by a dedicated fluidic device such as the one illustrated in FIG. 2. Fluids can thus be actively supplied, for example by means of a pump, syringe, etc. Alternatively, however, these exposure steps can be carried out without the need for a dedicated fluidic device. In this way, gases can be brought into contact with the functionalized surface 5 in a natural way, for example by simple diffusion or natural convection. In addition, the liquid(s) can be brought into contact with the functionalized surface 5 by introducing said surface (and here the prism) into the liquid in question. Similarly, the step of removing the liquid present on the functionalized surface 5 can be carried out by a dedicated fluidic device, as described below, or can be carried out by the user, for example by gravity flow, suction using a syringe, injection of a gaseous flow, by capillary pumping, etc.

In this example, the detection system 1 includes a fluid management device 20 adapted to successively expose the functionalized surface 5 to different fluids. In this example, the prior configuration of the optical measuring device 10 in ‘gas mode’ is carried out using the fluidic device 20. Of course, any other fluidic device can be used. Thus, in this example, the fluidic management device 20 is adapted to expose the functionalized surface 5 to a first gas G₁, called the reference gas, from a source such as here a reservoir or from the environment of the detection system 1 (in which case the reference gas G₁ can be ambient air). This reference gas G₁ is used to determine the working angle of incidence of the excitation signal generating surface plasmons, so that the detection system 1 exhibits optimum sensitivity. Note that the sensitivity of detection system 1 is here the relative variation of the measured signal, in this case the reflectivity R, in response to an adsorption or desorption event of a biological object. The reference gas G₁ can be dry air, humid air (e.g. room air), or any other gas such as argon or nitrogen.

The fluid management device 20 is also adapted to expose the functionalized surface 5, in a subsequent assimilation step, to a sample of interest E containing an aqueous carrier liquid in which the biological objects are present, from a source such as a reservoir here. As previously mentioned, the carrier liquid can be in a continuous or dispersed phase.

The fluidic management device 20 can be adapted to remove the sample of interest from the measuring chamber 2 and thus from the functionalized surface 5. This can be done in a rinsing step carried out after the assimilation step, during which the functionalized surface 5 is exposed to at least one rinsing liquid Lᵣ (buffer solution, detergent, ultrapure water, etc.) so that it flows over the functionalized surface 5. The rinsing liquid(s) Lᵣ allow the removal of any particles, salts or other substances present on the functionalized surface 5 and not specifically bonded to said surface, and capable of generating a spurious signal. The rinsing liquid(s) Lᵣ are preferably aqueous. It should be remembered here that specifically recognized biological objects remain bound to the ligands during this rinsing step, and are therefore not discharged from the measuring chamber 2. Alternatively, as indicated above, this removal step can be performed by the user and not by the fluidic device 20.

The fluidic management device 20 is adapted to remove any liquid remaining in contact with functionalized surface 5, whether liquid from the sample of interest or rinsing liquid, and to expose functionalized surface 5 to a second gas G₂. Preferably, as in the example shown in FIG. 2, the second gas G₁ is identical to the reference gas G₁, and can therefore be moist or dry air, or even argon or nitrogen. It can be introduced into the measuring chamber 2 either actively (e.g. gas injection) or passively (e.g. diffusion or natural convection). The biological objects are then in the environment of this second gas, in contact with the functionalized surface. Alternatively, as indicated above, this removal step can be performed by the user and not by the fluidic device 20.

Before presenting the detection method according to embodiments of the invention, we now detail, with reference to FIGS. 3A and 3B, a method for detecting biological objects which illustrates the need, in this case, for a detection system with improved performance, particularly in terms of spatial resolution and sensitivity.

Each of FIGS. 3A and 3B illustrate, on the left, a functionalized surface 5, and on the right, a curve showing the evolution of the reflectivity R associated with the measurement signal received as a function of the local refractive index at the functionalized surface. This curve is therefore equivalent to the classic SPR curve shown in FIG. 1E.

FIG. 3A illustrates a preliminary configuration of the optical measuring device 10, so that it generates surface plasmons at the functionalized surface 5 when said surface is exposed to a liquid (‘liquid mode’ configuration). Thus, the functionalized surface 5 is exposed to a reference liquid (e.g. a buffer solution) not containing the biological objects, and the value of the working angle θ_{R} is then chosen so that the optical measuring device 10 exhibits optimum sensitivity. This is detection by reflective interrogation, in the sense that the wavelength of the excitation signal and the working angle θ_{R} remain unchanged.

As the SPR curve shows, the reflectivity R measured at the sensitive sites has a value Rᵢ that is mainly associated with the refractive index n_{ref, I} of the reference liquid (and of course with that of the ligands). The Rᵢ value is substantially homogeneous at each sensitive site, which in this case can have a circular size with a diameter of around 300 μm. Alternatively, the reference liquid can be a buffer solution with refractive index n_{ref, I} equal to 1.33.

FIG. 3B illustrates an assimilation step in the biological object detection method, in which the functionalized surface 5 is exposed to the sample of interest, which in this case consists of the carrier liquid and the biological objects. The carrier liquid may be similar or identical to the reference liquid, and in this case is the same buffer solution. The biological objects have a refractive index n_{ob} that can be close to that of the carrier liquid, for example 1.4, whereas that of the carrier liquid is 1.33, a difference of 0.07. In addition, biological objects can be small, such as viruses with a size of around 100 nm, or bacteria with a size of around 1 μm. During this step, the biological objects bind to the ligands.

As indicated by the SPR curve, the reflectivity R measured at each sensitive site has two values, a localized value R_{ob} associated with the adsorbed biological objects, and a continuous value Rᵢ associated with the carrier liquid around the biological objects. In other words, in an image acquired by the optical measuring device 10, a spatially homogeneous background of value Rᵢ and smaller, brighter spots of value R_{ob} can be observed at each sensitive site.

It would appear, however, that detection system 1 must be particularly sensitive to be able to detect biological objects due to the very small difference, here equal to 0.07, between refractive indices n_{ob} and n_{ref, I}. Insufficient sensitivity would result in too small a variation in ΔR reflectivity, making it impossible to distinguish a measurement signal associated with the adsorption of a biological object from measurement noise (reflectivity variations not associated with the adsorption of a biological object). In other words, the contrast C, defined as the ratio (R_{ob}−Rᵢ)/(R_{ob}+Rᵢ) between the reflectivity R_{ob} measured at the biological object and the reflectivity Rᵢ measured in the sensitive site outside the biological objects, may be insufficient for effective detection of the biological objects.

Furthermore, the brighter R_{ob} reflectivity spots have dimensions close to those of the biological object, requiring a detection system with very high spatial resolution if we are to be able to detect each and every biological object adsorbed on a sensitive site for counting. In the case of a micrometer-sized bacterium, it is necessary to use a high spatial resolution detection system such as that described in the article by Laplatine et al. 2014 with a spatial resolution of the order of 2 μm. However, the use of such a detection system does not allow to distinguish individual biological objects when their size is well below spatial resolution, as is the case, for example, with viruses of around 100 nm in size.

In contrast to prior art methods, the method for detecting biological objects according to the invention allows for each of the biological objects adsorbed on a sensitive site to be detected by a detection system 1 with standard performance in terms of sensitivity and spatial resolution, even though the biological objects have a low refractive index difference with the carrier liquid and are small in size, preferably at most less than 200 times the spatial resolution of the detection system 1. By way of example, the detection method can detect and distinguish between viruses adsorbed on the same sensitive site, which have a size of the order of 100 nm, even though the spatial resolution of the detection system is of the order of 10 μm.

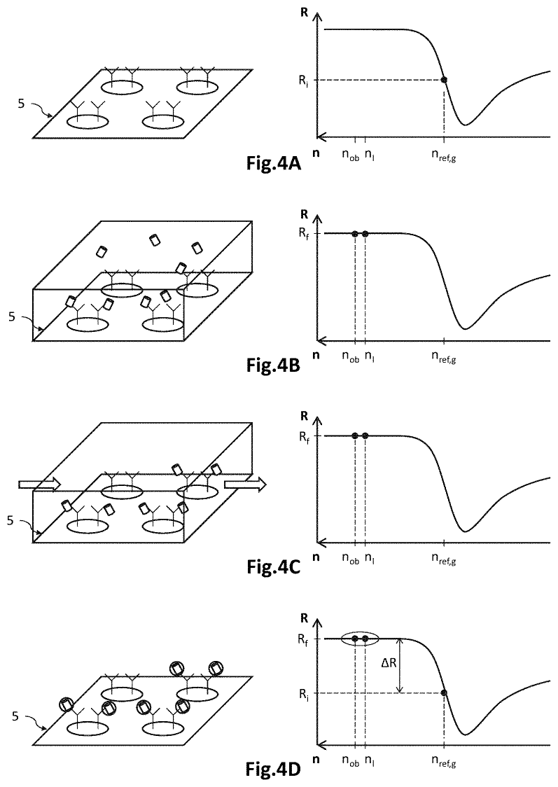

FIG. 4A to 4D illustrate steps in a detection method according to a first embodiment, using a detection system shown in FIGS. 1A and 1B and FIG. 2. In this example, the sample of interest containing the biological objects is a liquid sample.

FIG. 4A illustrates a preliminary configuration of the optical measuring device 10, so that it generates surface plasmons at the functionalized surface 5 when exposed to a gas (‘gas mode’ configuration). This configuration is said to be preliminary in the sense that it is carried out prior to the detection method. To achieve this, the functionalized surface 5 is exposed to a reference gas G₁ introduced into the measuring chamber 2. The reference gas can be introduced by the fluid management device 20, or by any other fluidic device. The reference gas G₁ therefore comes into contact with the functionalized surface 5. The reference gas G₁ does not contain biological objects, and preferably does not contain any elements that can bind ligands or generate a specific measurement signal. It has a refractive index n_{ref,g} equal to 1. The working angle θ_{R} is set to generate surface plasmons so that the detection system 1 has optimum sensitivity.

As the SPR curve shows, the reflectivity measured at the sensitive sites has an approximately spatially homogeneous Rᵢ value. It should therefore be noted here that the optical measuring device 10 of the detection system 1 used in the detection method according to the invention is configured in ‘gas mode’, unlike the detection system used in the detection method of FIGS. 3A and 3B where the optical measuring device is configured in ‘liquid mode’.

FIG. 4B illustrates an assimilation step in the detection method. For this purpose, the fluid management device 20 exposes the functionalized surface 5 to the sample of interest introduced into the measuring chamber 2. The sample of interest comprises an aqueous carrier liquid in which the biological objects are located. Here, the carrier liquid is in a continuous phase, so that the sample of interest is a liquid sample and not a mist or aerosol. The carrier liquid has a refractive index nᵢ equal to 1.33, for example, and may be a buffer solution. The biological objects have a refractive index n_{ob} here equal to 1.4, and can be, for example, a virus such as SARS-COV-2 with a size of around 100 nm. In this example, the spatial resolution of the measurement system is of the order of 10 μm, depending on the direction of propagation of the plasmon waves generated. During this step, the carrier liquid comes into contact with the functionalized surface 5, and the biological objects bind to the ligands.

As indicated by the SPR curve, the reflectivity R measured at each sensitive site has the same R_{f} value, substantially homogeneous over the entire spatial extent of the sensitive site, associated with both the carrier liquid of refractive index nᵢ and the biological objects of refractive index n_{ob}. This R_{f} value can be a maximum value of the SPR curve insofar as the difference Δn between refractive index nᵢ and refractive index n_{ref,g} is large, in this case of the order of 0.33. Of course, biological objects cannot be detected at this step.

FIG. 4C illustrates an optional, but advantageous, rinsing step. Rinsing consists of flowing a rinsing liquid, or several liquids in succession, over the functionalized surface 5, thus removing particles, objects, molecules etc. not specifically bound to the ligands and likely to induce measurement noise or spurious signals (variation in reflectivity not related to the biological objects to be detected). This improves detection quality and, in particular, the signal-to-noise ratio (SNR) by reducing spurious signal sources. For example, during the rinsing step, a buffer solution with a detergent can be injected, followed by a buffer solution identical to the carrier liquid (but obviously without the biological objects), and finally ultrapure water to remove any salts present in the buffer solution. It should be remembered that biological objects remain specifically adsorbed during this rinsing step, due to their strong affinity with the ligands and low dissociation.

FIG. 4D illustrates the next step in removing liquid from contact with the functionalized surface 5. To do this, the liquid present is removed from the measuring chamber 2, for example by gravity, suction, capillary action, etc., and the functionalized surface 5 is exposed to a second gas G₂, which obviously contains no biological objects. This second gas G₂ may be similar or identical to the reference gas used previously, particularly in terms of chemical composition, and has a refractive index close to or identical with n_{ref,g}. It preferably does not contain particles that affect the measurement signal. Here, too, the biological objects remain adsorbed during this removal step, so that they are located in the second gas that is in contact with the functionalized surface (and not in a liquid as in the detection method in FIGS. 3A and 3B). Preferably, the second gas G₂ is a moist gas and has a non-zero relative humidity, preferably at least 50%.

Following the removal step, the optical measuring device 10 acquires a detection image of the functionalized surface 5. The acquired image therefore includes images of each of the sensitive sites, and biological objects can be detected.

As the SPR curve shows, the reflectivity R measured at each sensitive site has two values, a localized value R_{f} associated with adsorbed biological objects, and a continuous value Rᵢ associated with the gas of refractive index n_{ref,g}. In other words, in the image acquired by the optical measuring device 10, a spatially homogeneous background of value Rᵢ and much brighter spots of value R_{f} can be observed at each sensitive site. The bright spots therefore have a very high intensity (reflectivity R), insofar as the value R_{f} of reflectivity R is close to or even equal to the maximum possible value (reflectivity saturation) of the SPR curve in the range of variation of the local refractive index n. In this way, the detection of biological objects is greatly improved, since the reflectivity difference ΔR, and therefore the contrast C, is increased compared with the situation in FIG. 3B, since the difference Δn between the refractive indices is now of the order of 0.33 instead of 0.07 (an increase of almost 400%).

In addition, the inventors have found that the light spots associated with biological objects have a spatial extent well in excess of the effective size (physical size) of the biological objects, for example, an area of the order of 15 to 20 μm in the case of SARS-COV-2 viruses whose effective size is of the order of 100 nm. This appears to be due to the presence of a thin film of water bound to each biological object, also known as the biological water layer or hydration layer, on the one hand, and to an optical phenomenon of scattering of the signal reflected by the biological object and its water film on the other hand. Remember that water molecules have an electrical polarity and can bind to biological objects such as viruses and bacteria. This would be biological water, as opposed to bulk water, which is discharged from the measuring chamber 2. Such a film of biological water is described in the article by Pal et al. entitled Biological water at the protein surface: Dynamical solvation probed directly with femtosecond resolution, PNAS vol. 99, no. 4, 1763-1768, 2002. This film of biological water may originate from the aqueous carrier liquid of the sample of interest, in particular when the rinsing step is not performed, and/or from the aqueous rinsing liquid(s). It can also come at least in part from water molecules in the second gas introduced during the removal step (this second gas G₂ then preferably has a relative humidity of at least 50%). As a result, it is possible to detect each and every biological object present at the same sensitive site, even though their size (e.g. of the order of 100 nm or 1 μm) is well below the spatial resolution (e.g. of the order of 10 μm) of the detection system.

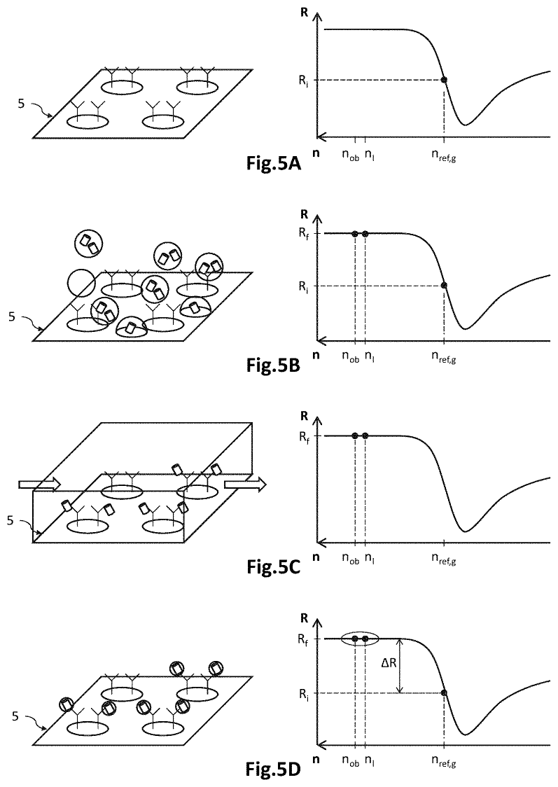

FIG. 5A to 5D illustrate the steps of a detection method according to a second embodiment using the detection system 1 configured in ‘gas mode’. In this example, the sample of interest containing the biological objects is a mist consisting of a gas (dispersing phase) and an aqueous carrier liquid in the form of droplets (dispersed phase) containing the biological objects. This procedure is similar to that described with reference to FIG. 4A to 4D, and only the different steps are detailed.

As shown in FIG. 5A, the optical measuring device 10 is configured in ‘gas mode’, as for the detection method according to the first embodiment (FIG. 4A), and not in ‘liquid mode’. The rinsing (FIG. 5C), removal and acquisition (FIG. 5D) steps are identical or similar to those described above. In contrast, during the assimilation step (FIG. 5B), where the functionalized surface 5 is exposed to the sample of interest, the latter is introduced into the measuring chamber 2 while the carrier liquid is in dispersed phase. In this way, droplets containing biological objects, for example with a volume of the order of a hundred cubic micrometers or more, are deposited on the functionalized surface 5, and the biological objects can bind to the ligands.

As illustrated by the SPR curve, the reflectivity R measured at each sensitive site can have two values, an R_{f} value associated with the droplets, and a continuous Rᵢ value associated with the gas (dispersing phase) of the sample of interest. In other words, on an image acquired by the optical measuring device 10, a spatially homogeneous background of value Rᵢ and larger, lighter spots of value R_{f} corresponding to droplets can be observed.

In this embodiment, a rinsing step can facilitate the removal of the liquid present in contact with the functionalized surface 5, in order to then carry out the image acquisition step enabling the detection of biological objects. It should be noted that, in this embodiment too, following the removal of any liquid present in contact with the functionalized surface 5, a film of bound biological water is present at the level of each biological object, allowing the biological objects to be distinguished from one another even though their size is less than the spatial resolution of the detection system.

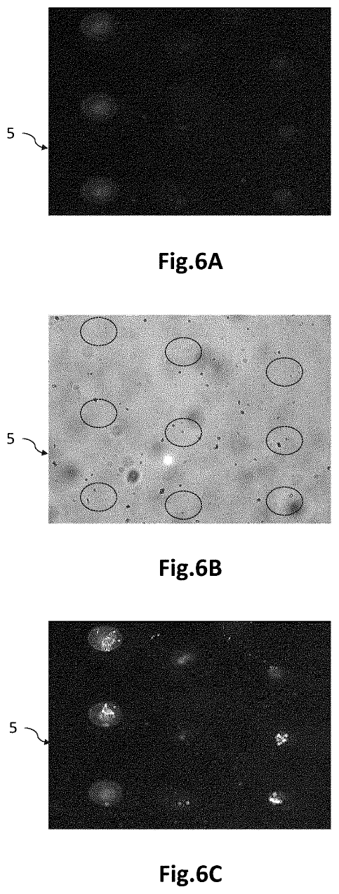

FIG. 6A to 6C are images of the functionalized surface 5 acquired at different steps of the detection method according to the first embodiment. In this example, the biological objects are inactivated SARS-COV-2 viruses.

FIG. 6A shows an image acquired as the reference gas G₁ is introduced into the measurement chamber (see FIG. 4A). This is upstream of the assimilation step of the detection method. The reference gas G₁ is ambient air. The functionalized surface 5 here comprises a matrix of 3 sets of 3 sensitive sites (delimited here by a dotted line), where the ligands are identical in one set but different from one set to another. The sensitive sites in the left and right columns contain positive control ligands, i.e. ligands adapted to bind specifically to viruses, in this case anti-S antibodies for the left column, and anti-S antibodies for the right column. The sensitive sites in the central column contain negative control receptors, i.e. receptors adapted not to bind specifically to viruses, in this case anti-KLH (Keyhole Limpet Hemocyanin) antibodies. It can be seen that, for the chosen working angle, the reflectivity R is very low for each sensitive site: we are therefore close to the reflectivity trough of the SPR curve. The contrast, defined here as the ratio (R_{in,moy}−Rₒᵤₜ)/(R_{in,moy}+Rₒᵤₜ) based on the R_{in,moy} reflectivity in a sensitive site and the Rₒᵤₜ reflectivity outside sensitive sites, is very low.

FIG. 6B shows an image acquired during the rinsing step, when a rinsing liquid (in this case ultrapure water) is in contact with the functionalized surface 5. As shown in FIG. 4C, reflectivity is very high over the entire functionalized surface 5. It is then impossible to distinguish sensitive sites, or of course viruses.

FIG. 6C shows an image acquired during the acquisition step, after any liquid in contact with the functionalized surface 5 has been removed. Note that the sensitive sites in the left-hand column show very bright spots (top and middle sites), as do the sensitive sites in the right-hand column (middle and bottom sites). In contrast, the sensitive sites of the central column (negative control) do not show very clear stains.

In line with FIG. 4D, reflectivity here has a minimum value Rₘᵢₙ outside sensitive sites; a value Rᵢ higher than this minimum value Rₘᵢₙ at sensitive sites but outside viruses; and a very high value R_{f}, close to a maximum value, where viruses are adsorbed. Thus, the contrast between R_{f} reflectivity associated with viruses and Rᵢ reflectivity associated with non-virus sensitive sites is very high and allows for the effective detection of viruses, but it is even higher between R_{f} reflectivity and Rₘᵢₙ reflectivity. Furthermore, the viruses appear here as very bright spots with a spatial extent of around 15 to 20 μm, in sensitive sites 300 μm in diameter, whereas SARS-Cov-2 viruses are around 100 nm in size. In this way, each virus can be distinguished from its neighbors, and the number of adsorbed viruses can be counted, even though the detection system has standard performance in terms of spatial resolution (in this case of the order of 5 to 10 μm). The correspondence between these light spots and the presence of viral particles was confirmed by scanning electron microscope (SEM) observation.

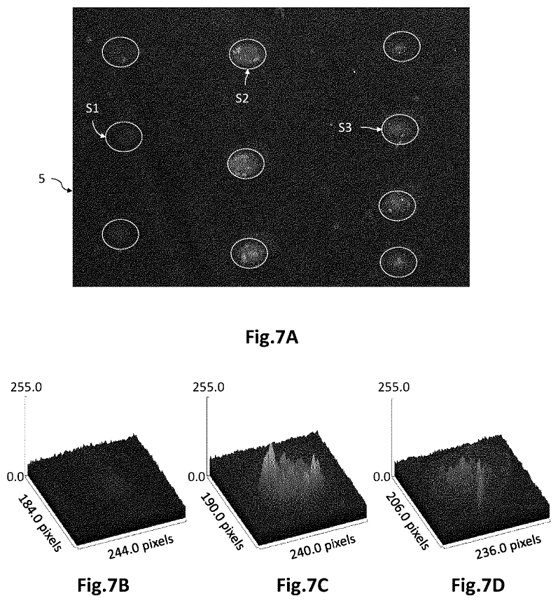

FIG. 7A and FIG. 7B to 7C are images of the functionalized surface 5, acquired following the removal step of the detection method according to the first embodiment. In this example, the biological objects are also inactivated SARS-COV-2 viruses.

FIG. 7A illustrates the functionalized surface where the sensitive sites in the left-hand column feature negative control receptors, here anti-KLH antibodies, those in the middle column feature positive control ligands, here anti-N antibodies, and those in the right-hand column feature positive control ligands, here anti-S antibodies.

It is noted that sites sensitive to anti-KLH antibodies do not show the very clear stains characteristic of the presence of viruses, whereas those with positive control antibodies do.

FIG. 7B shows a view of the S1 anti-KLH antibody-sensitive site. Reflectivity here varies on an 8-bit gray scale, i.e. between 0 and 255. Reflectivity has a very low homogeneous value, close to Rₘᵢₙ.

FIG. 7C shows a view of the anti-N antibody sensitive site S2. The reflectivity shows the minimum value Rₘᵢₙ outside the sensitive site, as well as peaks in the sensitive site representative of the presence of adsorbed virus. It is noticeable that the contrast is higher, and that the peaks have a much greater spatial extent than the effective size of the viruses.

FIG. 7D shows a view of the sensitive site S3 to SV205 anti-S antibodies. The reflectivity also shows some peaks representative of the presence of adsorbed viruses, but in smaller numbers than in FIG. 7C.

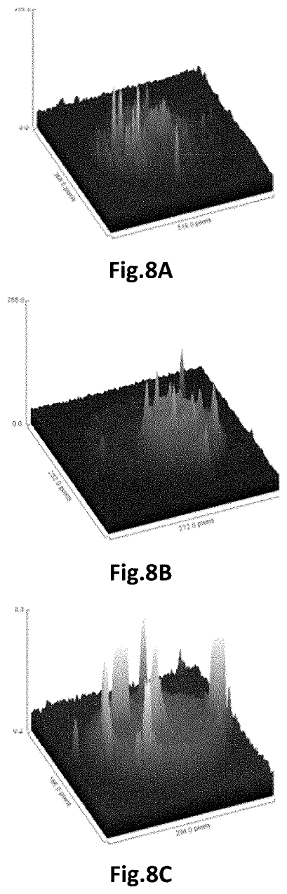

FIG. 8A to 8C are images of the functionalized surface, acquired following the removal step of the detection method according to the first embodiment, for different examples of biological objects. These images show the spatial evolution of reflectivity in 8-bit gray scale.

FIG. 8A shows an image of a sensitive site whose ligands are biotin. Here, the biological objects are streptavidin-modified polystyrene beads, about 200 nm in size. The reflectivity shows localized peaks representative of the adsorption of modified polystyrene beads, the intensity and spatial extent of which allow for detection and counting.

FIG. 8B shows an image of a sensitive site with anti-S ligands. The biological objects here are inactivated SARS-Cov-2 viral particles about 100 nm in size. The reflectivity also shows localized peaks representative of the presence of adsorbed viruses, the intensity and spatial extent of which allow for detection and counting. The change in the average reflectivity level associated with the sensitive site, outside of the peaks, may be due to the presence of soluble viral proteins recognized by the same ligands.

FIG. 8C shows an image of a sensitive site whose ligands are anti-E. coli. The biological objects here are E. coli K12 bacteria, about 1 to 2 μm in size. Here too, the reflectivity shows localized peaks representative of the presence of adsorbed bacteria, the intensity and spatial extent of which allow for detection and counting. Here, the average level of reflectivity, outside of the peaks, is associated with molecular fragments from the targeted bacteria.

As a result, the detection method according to the invention allows for efficient detection of each of the biological objects adsorbed on the sensitive sites, with a detection system offering standard performance in terms of spatial resolution and sensitivity. This is due in particular to the fact that, while the biological objects are brought into contact with the functionalized surface in a carrier liquid, the optical measuring device is previously configured in ‘gas mode’, and the acquisition of the detection image is carried out while the biological objects are in a gaseous environment.

Particular embodiments have just been described. Different variants and modifications will become apparent to a person skilled in the art.

1. A method for detecting biological objects, by means of a surface plasmon resonance imaging detection system comprising: a functionalized surface having at least one sensitive site formed of ligands adapted to bind to biological objects; and an optical measurement device configured to generate surface plasmon resonance at the functionalized surface when said surface is exposed to a gas, and adapted to acquire an image of the sensitive site; the method comprising:
an assimilation step, comprising exposure of the functionalized surface to a sample of interest formed of an aqueous carrier liquid containing the biological objects, the biological objects then binding to the ligands;
a step of acquiring an image of the sensitive site by the optical measuring device;
a step of detecting biological objects from the acquired image;
the method being wherein it comprises a step, carried out between the assimilation step and the acquisition step, of removing the liquid in contact with the functionalized surface, and of exposing the functionalized surface to a gas not containing the biological objects, the biological objects remaining bound to the ligands, the acquisition step then being carried out while the biological objects are bound to the ligands and the gas is in contact with the functionalized surface.

2. The method of detection according to claim 1, wherein the biological objects are smaller, preferably up to 200 times smaller, than a predefined spatial resolution of the detection system.

3. The method of detection according to claim 1, wherein the biological objects have a size between 50 nm and 50 μm.

4. The method of detection according to claim 1, wherein the detection system has a spatial resolution of at least 5 μm.

5. The method of detection according to claim 1, comprising a step of rinsing the functionalized surface with at least one liquid, carried out between the assimilation step and the removal step.

6. The method of detection according to claim 1, wherein, during the assimilation step, the liquid carrying the sample of interest is in a continuous or dispersed phase.

7. The method of detection according to claim 1, wherein, during the removal step, the gas has a relative humidity of at least 50%.

8. The method of detection according to claim 1, wherein the biological objects are chosen from viral particles, bacteriophages, bacteria and their spores, archaea, microscopic fungi and their spores, unicellular protozoa, blood or non-circulating cells, circulating vesicles, exosomes, pollens, biological objects comprising a synthetic particle to which at least one ligand or biological protein is attached.
